(19) United States
(12) Patent Application Publication
(10) Pub. No.: US 2016/0008026 A1
(43) Pub. Date: Jan. 14, 2016

(54) APPARATUS AND METHODS INVOLVING ELONGATED-MEDICAL INSTRUMENT FOR SENSING TISSUE INTERACTION FORCES

(71) Applicant: THE BOARD OF TRUSTEES OF THE LELAND STANFORD JUNIOR UNIVERSITY, Palo Alto, CA (US)

(72) Inventors: Santhi Elayaperumal, San Francisco, CA (US); Jung Hwa Bae, Stanford, CA (US); Bruce L. Daniel, Palo Alto, CA (US); Mark R. Cutkosky, Palo Alto, CA (US)

(21) Appl. No.: 14/772,754

(22) PCT Filed: Mar. 4, 2014
(86) PCT No.: PCT/US2014/020369

§ 371 Date: Sep. 3, 2015

Related U.S. Application Data
(60) Provisional application No. 61/772,061, filed on Mar. 4, 2013.

(51) Int. Cl. A61B 17/34; A61B 5/01; A61B 19/00

(52) U.S. Cl. CPC A61B 17/3403; A61B 19/2203; A61B 5/01; A61B 2019/465; A61B 2019/5261; A61B 2017/00911

(57) ABSTRACT

Various aspects as described herein are directed to methods and systems that include a tissue-engagement apparatus. The tissue-engagement apparatus includes a distal needle portion having a sharp-end region to be applied to a tissue surface. The tissue-engagement apparatus also includes a proximate needle portion to attach to a needle base, and an elongated needle portion, situated between the distal needle portion. The elongated needle portion includes a plurality of openings that accentuate haptic-type forces carried by the elongated needle portion in response to engagement between the sharp-end region and the tissue surface. Additionally, the proximate needle portion includes a communication pathway that conveys information, from the distal needle portion along the elongated needle portion, which characterizes forces due to the engagement between the sharp-end region and the tissue surface.

### FEDERALLY-SPONSORED RESEARCH AND DEVELOPMENT

This invention was made with Government support under contract CA159992 awarded by the National Institutes of Health. The Government has certain rights in this invention.

### OVERVIEW

Various aspects of the present disclosure relate to force sensing devices, methods and systems that include tip/force sensing needles. Such sensing needles are sometimes used in the form of a surgical tool, such as a needle or scalpel, by doctors or surgeons, and other interventionists. The “sensing” aspect(s) of the needles convey tactile or haptic information by the sensations at or near the end of the surgical tool. As one of many examples, a physician can use a needle to sense the difference between various healthy tissues and cancerous tissues when scraping or removing tissue during a biopsy procedure. Similarly, when a physician punctures a membrane or hits an obstacle while inserting a needle through tissue, haptic information can be conveyed up the needle to the physician's hand.

Procedures involving medical robots can also be benefited by such haptic forces including tactile cues indicative thereof. An arm of a medical robot is a form of surgical tool that, if properly equipped and implemented, can employ technology to sense its own configuration in space and, in some instances, also sense forces at the mechanical wrist of the robot. However, in most cases, sensors are not employed on the inserted tool itself. The sensing apparatus presented in this disclosure enables for force sensing capabilities at the tool's most distal-end.

### SUMMARY

Various aspects of the present disclosure are directed toward object-engagement apparatuses, each having a distal portion with a sharp-end region that is applied to a surface of the object. The apparatus includes a proximate portion that attaches to a base and an elongated portion, situated between the distal portion and the proximate portion. The elongated portion includes openings that accentuate haptic-type forces carried by the elongated portion, in response to engagement between the sharp-end region and the object surface. Further, the elongated portion also includes a communication pathway that conveys information, from the distal portion along the elongated portion, which characterizes forces due to the engagement between the sharp-end region and the surface.

More specific aspects of the present disclosure are directed to the context of biological applications involving a tissue-engagement apparatus. The tissue-engagement apparatus includes a distal needle portion having a sharp-end region that is applied to a tissue surface. The tissue-engagement apparatus includes a proximate needle portion that attaches to a needle base. Further, the tissue-engagement apparatus includes an elongated needle portion, situated between the distal needle portion and the proximate needle portion. The elongated needle portion includes openings that accentuate haptic-type forces carried by the elongated portion, in response to engagement between the sharp-end region and the tissue surface. Further, the distal needle portion also includes a communication pathway that conveys information, from the distal needle portion along the elongated needle portion, which characterizes forces due to the engagement between the sharp-end region and the tissue surface.

Various aspects of the present disclosure are also directed toward methods that include a tissue-engagement apparatus. The methods include providing a distal needle portion having a sharp-end region that is to be applied to a tissue surface, a proximate needle portion attached to a needle base, and an elongated needle portion, situated between the distal needle portion and the proximate needle portion. The elongated needle portion includes openings that accentuate loads carried by the elongated portion in response to engagement between the sharp-end region and the tissue surface. Additionally, the proximate needle portion includes a communication pathway that conveys information, from the distal needle portion along the elongated needle portion, which characterizes forces due to the engagement between the sharp-end region and the tissue surface. The methods also include applying the sharp-end region to the tissue surface.

The above discussion/summary is not intended to describe each embodiment or every implementation of the present disclosure. The figures and detailed description that follow also exemplify various embodiments.

### FIGURES

Various example embodiments may be more completely understood in consideration of the following detailed description in connection with the accompanying drawings.

FIG. 1 shows an example tissue-engagement apparatus, consistent with various aspects of the present disclosure;

FIG. 2 shows an example tissue-engagement apparatus and inset cross-section of the example tissue-engagement apparatus, consistent with various aspects of the present disclosure;

FIG. 3 shows another view of an example tissue-engagement apparatus, consistent with various aspects of the present disclosure;

FIG. 4 shows an example operation of a fiber Bragg gratings (FBG) sensor, consistent with various aspects of the present disclosure;

FIG. 5 shows another example tissue-engagement apparatus and inset cross-section of the example tissue-engagement apparatus, consistent with various aspects of the present disclosure;

FIGS. 6A and 6B show example finite element analysis (FEA) results for axial strain on another example tissue-engagement apparatus, consistent with various aspects of the present disclosure;

FIGS. 7A and 7B show example FEA results for axial strain on another example tissue-engagement apparatus, consistent with various aspects of the present disclosure;

FIG. 8 shows another example tissue-engagement apparatus needle connected to a 6-axis force/torque sensor with handle for insertion experiments, consistent with various aspects of the present disclosure;

FIG. 9 shows example axial FBG data from a needle sharp-end region compared to force data from a needle base during tap testing, consistent with various aspects of the present disclosure;

FIG. 10 shows example axial FBG data from a needle tip compared to force data from needle base during insertion in phantom; consistent with various aspects of the present disclosure.

FIG. 11A shows an example microscope image of a needle tip and groove of a tissue-engagement apparatus, consistent with various aspects of the present disclosure;

FIG. 11B shows an example microscope image of openings of a tissue-engagement apparatus, consistent with various aspects of the present disclosure;

FIG. 12 shows an example plot of a wavelength shift to applied force for a FBG sensor at the needle tip of a tissue-engagement apparatus, consistent with various aspects of the present disclosure; and

FIG. 13 shows an example plot of frequency response to axial loading of a tissue-engagement apparatus, consistent with various aspects of the present disclosure.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the scope of the disclosure including aspects defined in the claims.

### DESCRIPTION OF EXAMPLE EMBODIMENTS

Various aspects of the present disclosure are directed towards object-engagement apparatuses, where the target of such engagement includes tissue and other elongated or needle-like structures in which tactile and/or vibration forces can be carried in response to the engagement. While not necessarily so limited, aspects of the present disclosure are discussed in the example context of apparatus (e.g., devices, tools and systems) and methods involving a tissue-engagement tool. Certain other aspects of the present disclosure are directed toward sensor technology to resolve axial and radial forces, as well as compensate for temperature effects at an end region when the apparatus is inside the tissue region. In certain embodiments, a tissue-engagement apparatus includes openings (e.g., oval holes) along a longitudinal section of a tissue-engagement apparatus (such as a needle). The tissue-engagement apparatus can include a sharp-end region, for example, at the tip of a needle in embodiments where the tissue-engagement apparatus is a surgical needle; whereas for other surgical instruments, the sharp-end region need not be limited to the tip.

Various aspects of the present disclosure are directed toward tissue-engagement apparatuses. One such tissue-engagement apparatus includes a distal needle portion having a tip that is applied to, and also sometimes through, a tissue surface. Application to a tissue surface can include applying an end of the apparatus across the textured material or through a heterogeneous medium. As examples, the tissue surface can include the skin of a patient, subcutaneous tissue, and membranes in inner organs, blood vessels and serosa. The surfaces can also include membranes in inner organs, blood vessels and serosa. The tissue-engagement apparatus can sense membranes deep inside the body (without limit) as well as surface contact. Additionally, the tissue-engagement apparatus can be used during tissue insertion. Forces acting on the end region of the tissue-engagement apparatus can include both friction along a longitudinal portion of the tissue-engagement apparatus, and cutting loads at the end region of the tissue-engagement apparatus which also includes a proximate needle portion that attaches to a needle base. Further, the tissue-engagement apparatus includes an elongated needle portion, situated between the distal needle portion and the proximate needle portion.

In certain embodiments, the elongated needle portion includes openings that accentuate haptic-type forces carried by the elongated portion in response to engagement between the end region and the tissue surface. Haptic-type forces include forces that can be felt. In certain specific embodiments, the elongated needle portion also includes a communication pathway that conveys information, from the distal needle portion along the elongated needle portion, that characterizes forces due to the engagement between the end region and the tissue surface.

The communication pathway is implemented, in certain embodiments, to secure at least one fiber optic line, which conveys optical information, and the distal needle portion and the elongated needle portion are contiguous parts of a needle. Additionally, the tissue-engagement apparatus also includes the needle base, and at least one FBG sensor. The FBG sensor(s) is arranged along the elongated needle portion, and is configured with at least one fiber optic line to convey at least one of transverse load information, axial load information and temperature information. Other embodiments of the present disclosure also include at least one sensor that measures optical wavelength shifts, communicated by the communication pathway, that occur due to the forces at the end region due to a compressive load on the end region, and the elongated needle portion due to engagement between the end region and the tissue surface. In certain embodiments, the information conveyed by the communication pathway includes temperature inside the tissue surface, and three-dimensional quantification of bending, and tensile and compressive forces along the length of the elongated needle member.

The end region of the tissue-engagement apparatus can be configured as a trocar tip. Additionally, the tissue-engagement apparatus can be constructed with materials known to be compatible with magnetic resonance imaging (MRI), and the communication pathway defines one or more grooves constructed along the elongated needle portion (each of the grooves secures a fiber optic line). Additionally, the tissue-engagement apparatus can also include at least one groove in and along the elongated needle portion, between the distal end portion and the proximate end portion. The groove houses the communication pathway. Further, in other embodiments, the communication pathway conveys information indicative of a vibration of the elongated needle portion. In certain embodiments, the openings are located closer to the distal needle portion than to the proximate needle portion. Further, the end region is configured to puncture the tissue surface, and the communication pathway conveys information including information indicative of a vibration of the elongated needle portion and light.

Various aspects of the present disclosure are also directed toward methods that include a tissue-engagement apparatus. The methods include providing a distal needle portion having an end region that is to be applied to a tissue surface, a proximate needle portion attached to a needle base, and an elongated needle portion, situated between the distal needle portion and the proximate needle portion. The proximate needle portion includes openings that accentuate loads carried by the elongated portion in response to engagement between the end region and the tissue surface. Additionally, the proximate needle portion includes a communication pathway that conveys information, from the distal needle portion along the elongated needle portion, to characterize forces ensuing from engagement between the end region and the tissue surface. The methods also include applying the end region to the tissue surface.

In certain more specific embodiments, applying the end region to the tissue surface includes determining the information via at least one FBG sensor arranged along the elongated needle portion and communicatively coupled to the communication pathway.

The embodiments and specific applications discussed herein may be implemented in connection with one or more of the above-described aspects, embodiments and implementations, as well as with those shown in the appended figures.

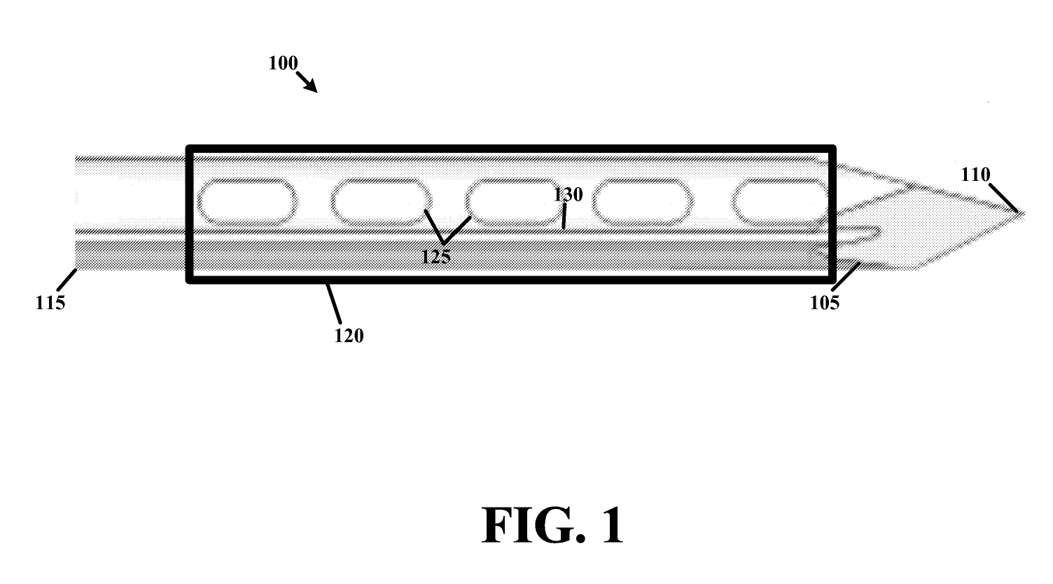

Turning now to the figures, FIG. 1 shows an example tissue-engagement apparatus 100, consistent with various aspects of the present disclosure. The tissue-engagement apparatus 100 includes a distal needle portion 105 having an end region 110 that is to be applied to a tissue surface. The end region 110 can be a sharp-end region such as the tip of a needle. Additionally, the tissue-engagement apparatus 100 includes a proximate needle portion 115 that can attach to a needle base. Further, the tissue-engagement apparatus 100 includes an elongated needle portion 120, situated between the distal needle portion 105 and the proximate needle portion 115. The elongated needle portion 120 includes a plurality of openings 125 that accentuate haptic-type forces carried by the elongated needle portion 120 in response to engagement between the end region 110 and the tissue surface. The elongated needle portion 120 also includes a communication pathway 130 that conveys information, from the distal needle portion 105 along the elongated needle portion 120. The information characterizes forces due to the engagement between the end region 110, and/or an illustrated shoulder region immediately adjacent thereto, and the tissue surface.

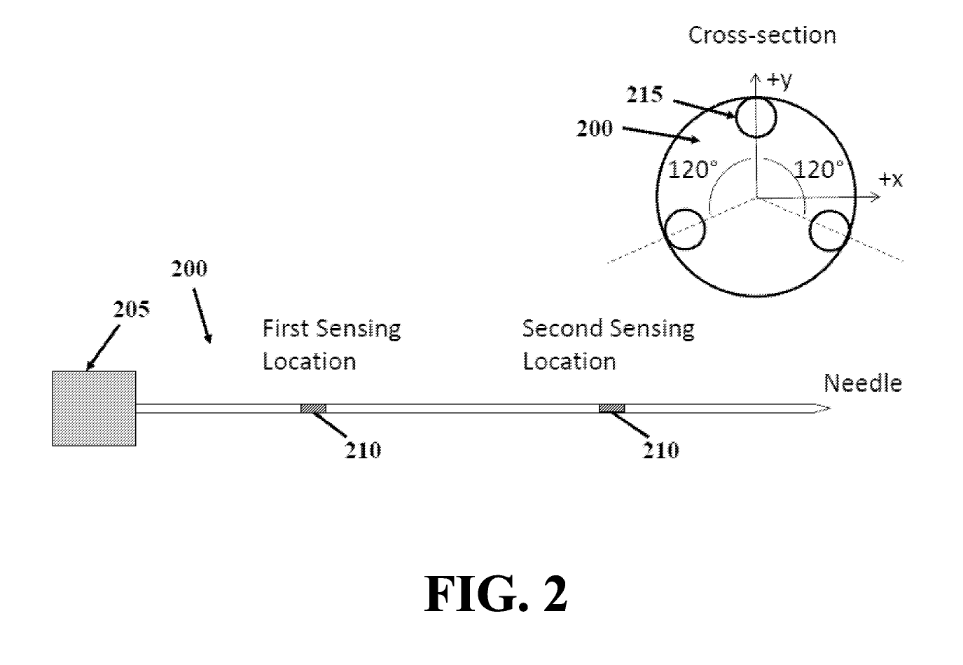

FIG. 2 shows an example tissue-engagement apparatus 200 and inset cross-section of the example tissue-engagement apparatus 200, consistent with various aspects of the present disclosure. The tissue-engagement apparatus 200 shown in FIG. 2 is a needle. Additionally, the tissue-engagement apparatus 200 is shown connected to a needle base 205 (e.g., the proximate needle portion of the tissue-engagement apparatus 200). The tissue-engagement apparatus 200 includes at least one sensor 210. For instance, as shown in FIG. 2, sensors 210 are located at sensor location 1 and sensor location 2 (including 3 sensors at each location), along an elongated portion of the tissue-engagement apparatus 200. A greater number of sensors can be present in certain embodiments of the present disclosure.

In certain embodiments, the tissue-engagement apparatus 200 utilizes sensors 210 that are FBG sensors. The FBG sensors are optically-based. As shown in the inset of FIG. 2, the tissue-engagement apparatus 200 includes three optical fibers 215. The optical fibers 215 are coupled to the sensors 210, and can be embedded symmetrically in the tissue-engagement apparatus 200. In the example shown, the optical fibers 215 are embedded 120 degrees apart.

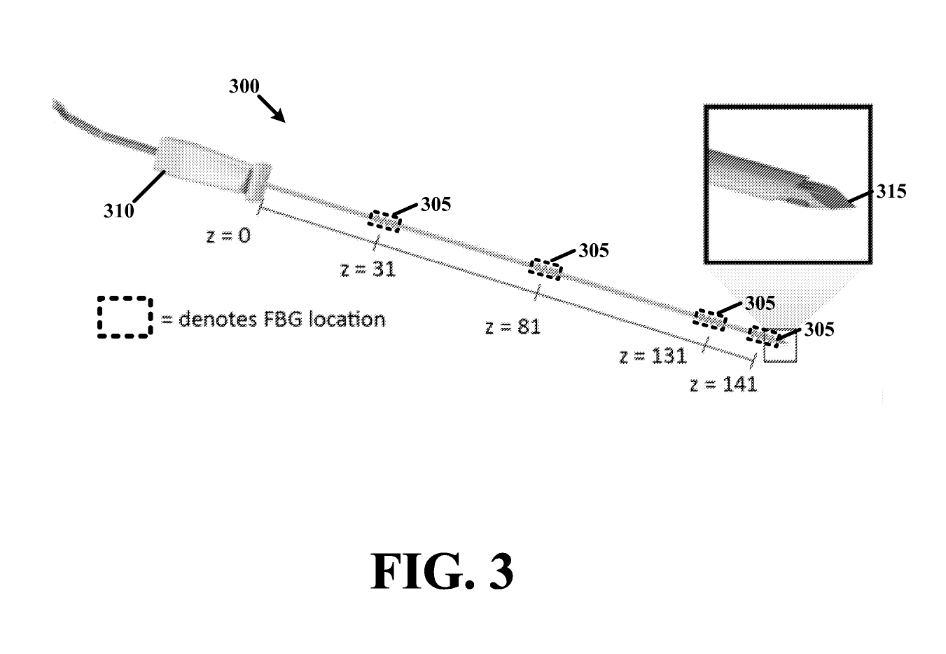

FIG. 3 shows another view of an example tissue-engagement apparatus 300, consistent with various aspects of the present disclosure. The tissue-engagement apparatus 300 includes triplets of FBG sensors 305 located at various locations along the tissue-engagement apparatus 300. In the example of FIG. 3 (presented for illustrative purposes), there are four locations along the tissue-engagement apparatus 300: at 31 mm, 81 mm, 131 mm and 141 mm, as measured from the base 310 of the tissue-engagement apparatus 300. In this manner, the FBG sensors 305 are set apart to approximate a full curvature profile of the tissue-engagement apparatus 300. Additionally, the middle of the last FBG sensor is centered over the holes in the tissue-engagement apparatus 300, as shown in FIG. 1, for example, to measure loads at a sharp-end region 315 of the tissue-engagement apparatus 300 where bending moments and strains are comparatively small. As described in further detail below, the FBG sensors 305 are used to measure bending of the tissue-engagement apparatus 300. Additionally, because FBG sensors are sensitive to temperature variations, the temperature at the sharp-end region 315 is measured by one of the FBG sensors 305.

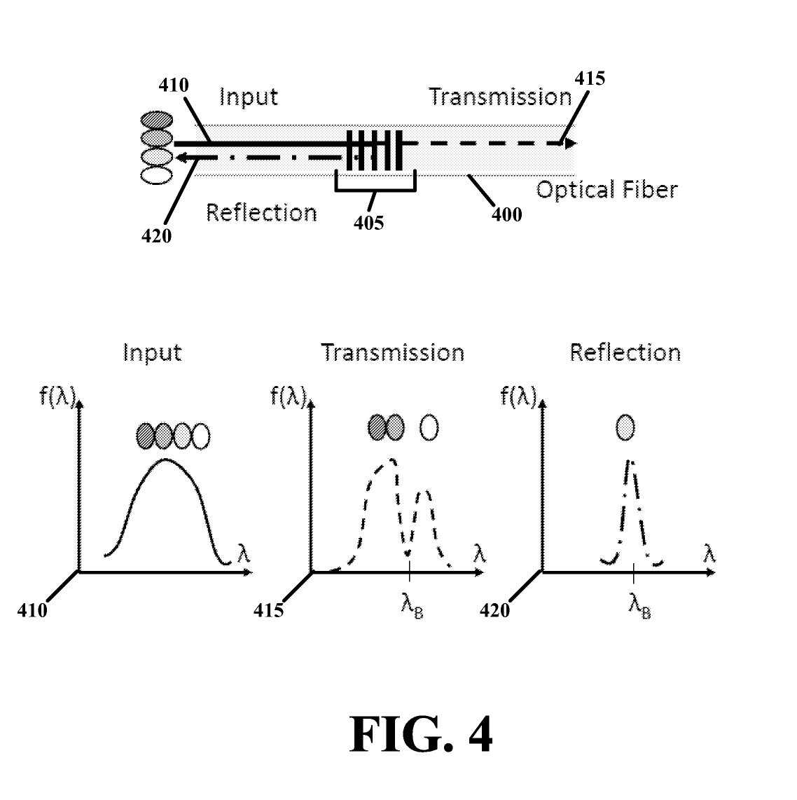

FIG. 4 shows an example operation of a FBG sensor, consistent with various aspects of the present disclosure. Optical fiber 400 used with FBG 405 measure optical wavelength shifts corresponding to strains. The wavelength shifts occur in response to a light source providing light (input 410) along the optical fiber 400. Light is transmitted (transmission 415) through the FBGs 405. For sensing mechanical and thermal strains, the FBG sensors use the light reflection of specific wavelengths (reflection 420) that shift proportional due to the strain to which the sensor is subjected. Measurement of these wavelength shifts provides the basis for strain and temperature sensing.

More specifically, both the fiber's effective refractive index, η_{eff}, and the grating period, Λ, vary with changes in strain, ε, and temperature, ΔT. The center Bragg wavelength λ_{B} is

λ_{B}=2η_{eff}Λ (1)

For FBG sensors made of isotropic materials, the wavelength shift due to mechanical and thermal strains is

Δλ_{B}=(1−Pₑ)(ε_{z}+αΔT)λ_{B}+ζΔT (2)

where Pₑ is the equivalent photoelastic coefficient, ε_{z} is axial strain, ζ is the thermo-optic coefficient of the FBG and α is the thermal coefficient of expansion of the material to which the FBG is bonded. For an FBG centered around 1550 nm, example values are η_{eff}=1.51, Pₑ=0.22, α=0.55e-6/° C., and ζ=10 pm/° C. for silica fiber. With the appropriate optical interrogator, thermal compensation and calibration, small strains, on the order of 0.1μ strain, can be measured at speeds in the kHz range.

The actual wavelength changes due to strain and temperature depend on the substrate and configuration in which the FBGs are adhered. The wavelength shift due to strain and temperature is often simplified as:

Δλ_{B}=K_{ε}ε+K_{T}ΔT (3)

where K_{ε} and K_{T} are constants representing the sensitivity to mechanical strains and temperature variations, respectively.

Bending strains and axial forces that result from forces applied to a tissue-engagement apparatus can also be measured. As an illustrative example, a tissue-engagement apparatus has an FBG positioned at the midpoint (½) of a tissue-engagement apparatus's length. Modeling the tissue-engagement apparatus as a cantilever beam with a circular cross-section, if a tip force of magnitude fᵣ is radially applied (normal to the apparatus's neutral axis), the strain at the FBG is

$${ɛ}_{b} = \frac{{M}_{C}}{EI} \approx \frac{2 {f}_{r} l}{π {r}^{3} E} \qquad ( 4 )$$

where M is the moment produced by fᵣ, c is the radial distance from the neutral axis of the needle to the FBG center (slightly less than r in the maximum case), I is the area moment of inertia and E is the Young's modulus of the beam material. If a load is applied axially to the tip of the apparatus, the strain is

$${ɛ}_{a} = \frac{{f}_{z}}{E π {r}^{2}} \qquad ( 5 )$$

For the case that fᵣ=f_{z}, with needle dimensions r=0.5 mm and I=150 mm, the ratio of strains is εₐ/ε_{b}= 1/600. In addition, there is a problem that axial and thermal strains produce exactly the same effects on a cylindrical beam with a symmetric arrangement of sensors. A solution to overcome this coupling issue is to locate additional FBG sensors near the needle tip, and to modify the tip geometry, making it asymmetric and increasing the strains resulting from axial forces.

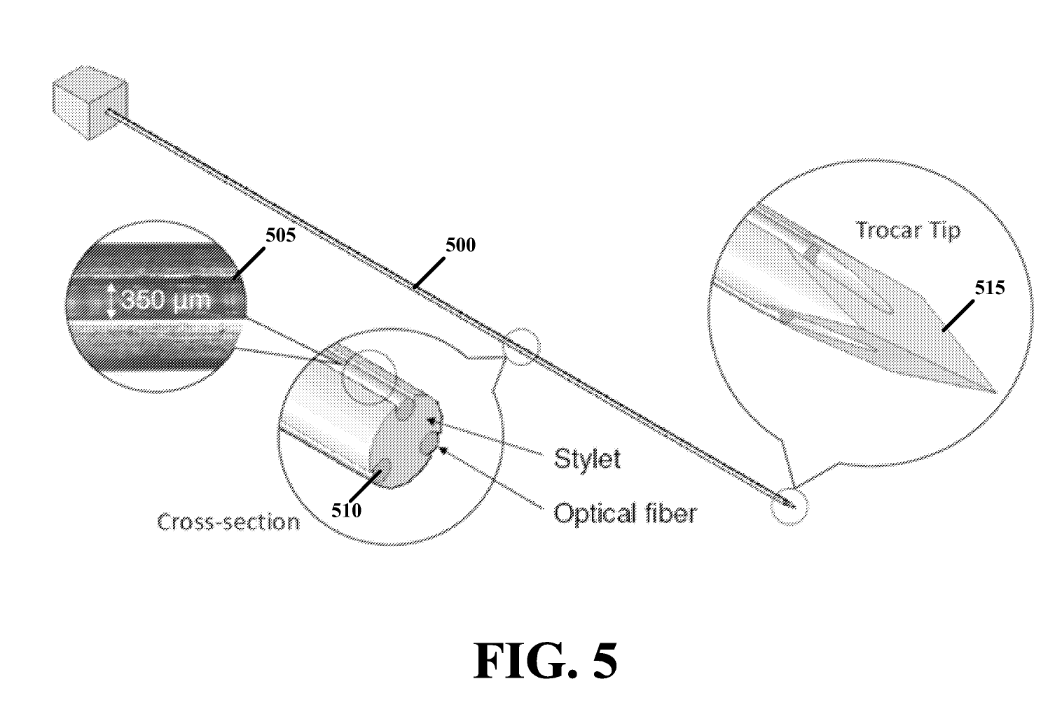

FIG. 5 shows another example tissue-engagement apparatus 500 and inset cross-section of the example tissue-engagement apparatus, consistent with various aspects of the present disclosure. The tissue-engagement apparatus 500 includes grooves 505 along an elongated portion of the tissue-engagement apparatus 500. The grooves 505 house optical fibers 510 in the tissue-engagement apparatus 500. As described in detail above, the optical fibers 510 are connected to FBG sensors for measuring optical wavelength shifts that correspond to strains on the tissue-engagement apparatus 500. Additionally, the example tissue-engagement apparatus 500 shown includes a trocar tip 515 that is provided to pierce tissue (and underlying tissue).

In certain embodiments of the present disclosure, a cross-section of the tip of the tissue-engagement apparatus is asymmetrical as a result of the placement of oval openings/holes. This asymmetry is useful in decoupling affects due to thermal strain and mechanical strain from axial loads. Additionally, the size of features and sensing elements (FBG sensors) are small such that the sensing elements can fit inside a needle of less than 1 mm in diameter. Further, the FBG sensors are immune to electro-magnetic interference, and thus, are MRI-compatible. The MRI-compatibility is due to a light source and optical interrogating electronics being kept outside the scanner suite. In addition, the sensors have very high precision, the ability to sense micro-strains, and sampling can be achieved in the kHz range.

Additionally, in certain embodiments of the present disclosure, the tissue-engagement apparatus is used in minimally invasive procedures performed with a needle, including biopsy, brachytherapy, and cryosurgery and other forms of ablation, as well as puncture of blood vessels, cysts, the thecal sac and other fluid-filled hollow structures. Further, the tissue-engagement apparatus can be, used in image guided interventions including Ultrasound, MRI and CT. Additionally, as noted above, the tissue-engagement apparatus can be used with industrial robotic applications where small-scale force sensing technologies are needed. Further, the tissue-engagement apparatus, in certain embodiments, is used in haptic-feedback applications, including probing surfaces either directly or remotely via a teleoperated device. Further, the tissue-engagement apparatus can be used to measure the stiffness of materials, especially of materials embedded in other materials, and measure dynamic forces such as those that occur during membrane puncture, texture recognition and obstacle encounters.

The tissue-engagement apparatus can be manufactured using a variety of different methods. For instance, the grooves and openings/holes of the tissue-engagement apparatuses, consistent with various aspects of the present disclosure can be formed by electric discharge machining, laser cutting, waterjet cutting, micro milling and material extrusion.

Additionally, in certain embodiments, rather than using FBG sensors, foil strain gauges/strain gauge rosettes, other resistive or capacitive sensors and other optical strain/bend/flex sensors can be used.

### Experimental Results and Detailed Embodiments

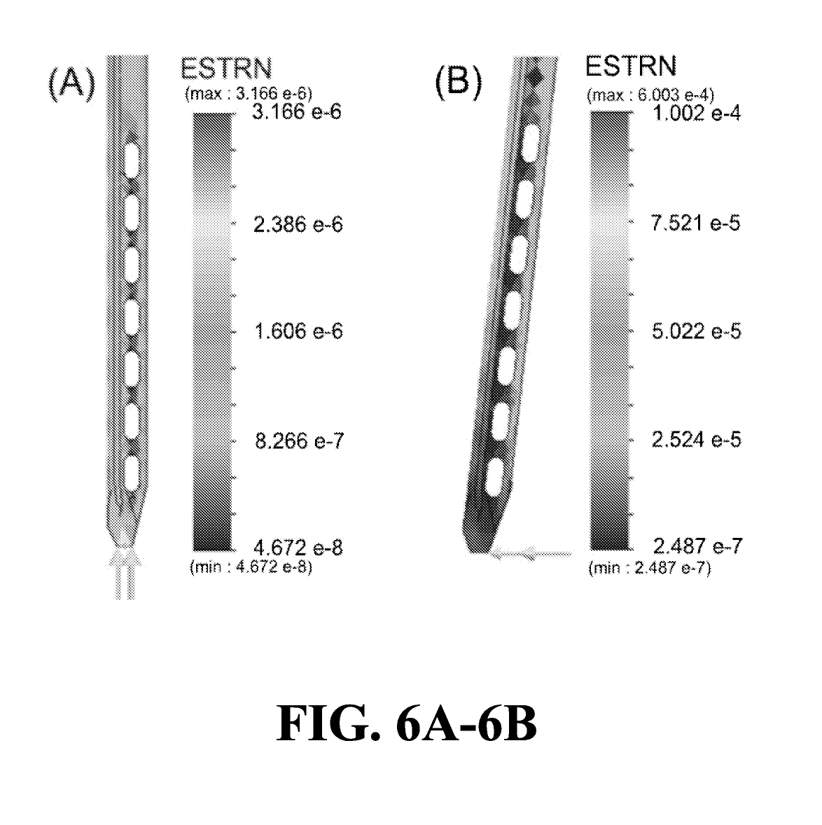

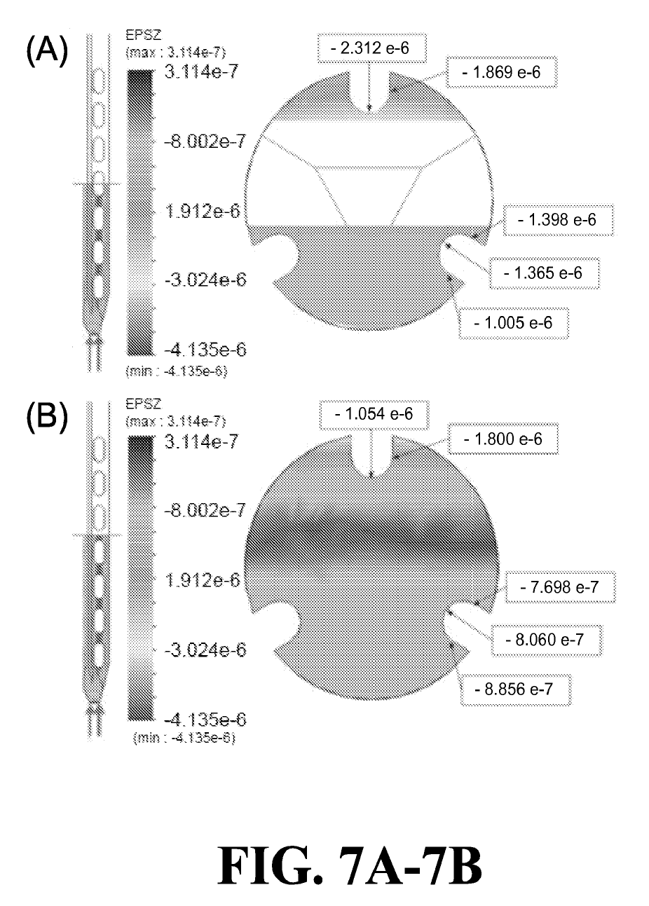

FIGS. 6A and 6B show example FEA results for axial strain on another example tissue-engagement apparatus, consistent with various aspects of the present disclosure. FIGS. 7A and 7B show a close-up example FEA results for axial strain on another example tissue-engagement apparatus, consistent with various aspects of the present disclosure. For purposes of the FEA, the tissue-engagement apparatus used was a “blunted” needle to more realistically apply force to nodes at the tip. FIGS. 6A-6B and 7A-7B show the FEA results for strain under 0.1 N axial and radial loads. As can be seen, the distal FBGs experience somewhat increased strains due to radial forces. However, the main difference with respect to a needle without openings/holes is in the axial response. The comparative strains for the grooved needle with and without holes, measured along the top groove over a 1.5 mm length at the center of the FBG, are summarized in Table 1.

TABLE 1
Average Axial Strains at Upper FBG Location

| Load Applied | ε_{avg} needle with | | Ratio |
|---|---|---|---|
| At Tip | holes | ε_{avg} plain needle | (modified:plain) |
| F_{y} = 0.1N | 3.145e−5 | 1.817e−5 | 1.73 |
| F_{z} = 0.1N | −1.837e−6 | −6.02e−7 | 3.05 |

As expected, the top FBG is more sensitive to loads in the y direction (vertical in FIG. 7A-B) than in the x direction (horizontal in FIG. 7A-B). For a purely axial force, the increase in strain compared to a needle without holes is approximately 300%. The sensitivity to axial loads is still less than for radial forces in the y direction (by a factor of approximately 1/17), but is much improved over the 1/600 sensitivity ratio at the middle of a tool. The yield stress of MP35N at 0.2% strain is 379 MPa. Using the equation for bending stress at the needle base:

$${σ}_{b} = \frac{{M}_{C}}{I} \approx \frac{4 {f}_{c} l}{π {r}^{3}} \qquad ( 6 )$$

the critical load for the needle is approximately f_{c}=0.2 N for radial loads. A factor of safety analysis on the FEA model showed a critical load of 0.21 N to cause yielding at the needle base. Under this load, maximum stresses at the region with the holes were ≈91 MPa, which is well under the yield stress. Therefore, the strength of the needle is not reduced by the addition of the holes. Additionally, adding holes did not make the needle tip more susceptible to buckling than a solid design. FEA buckling analysis showed that the ratio of critical load for buckling a needle with holes versus a plain needle was 0.9991.

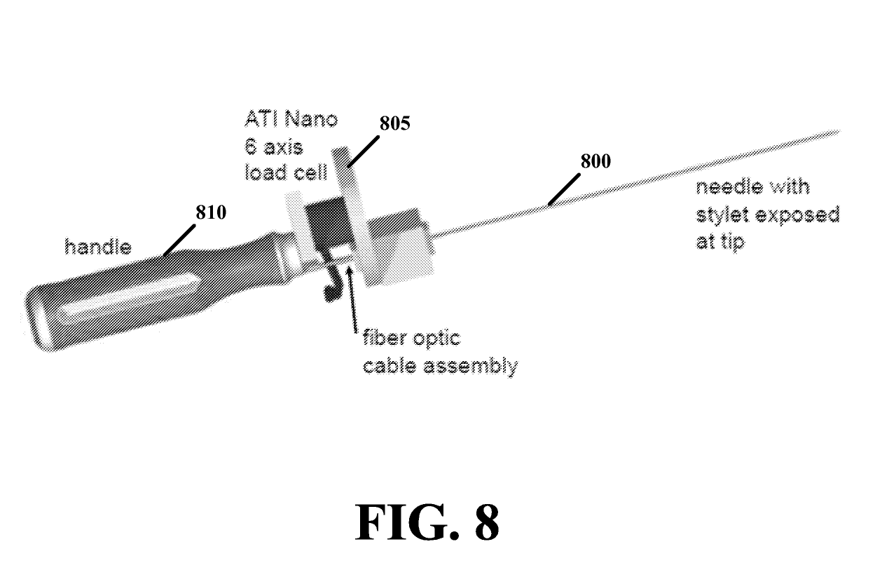

FIG. 8 shows another example tissue-engagement apparatus needle 800 connected to a 6-axis force/torque sensor 805 with a handle 810 for insertion experiments, consistent with various aspects of the present disclosure. The test of the utility of the needle 800 is to compare measured tip forces with those that could be sensed at the base, directly with a physician's hand. For this comparison, the needle 800 was affixed to a small 6-axis force/torque sensor 805 (ATI Nano 175), which was mounted to a handle 810. With this apparatus it is possible for a user to insert the needle into tissue phantoms, while recording forces from the needle tip using the FBG sensors and from the needle base using the force/torque sensor.

To show correlation between the FBG data and the force/torque sensor data, the handle assembly 810 was first used to tap on a sample of urethane rubber (shore 60 A durometer) in a water bath. The needle tip was pressed against the rubber, tapped three times and lifted completely off the rubber three times. The initial non-contact readings from both the force/torque sensor 805 and the needle were subtracted from the readings during contact. For the needle, the wavelength common mode (i.e., the average wavelength shifts for the three distal FBGs) gives the wavelength change due to axial loading for comparison with the measured F_{z} force from the force/torque sensor.

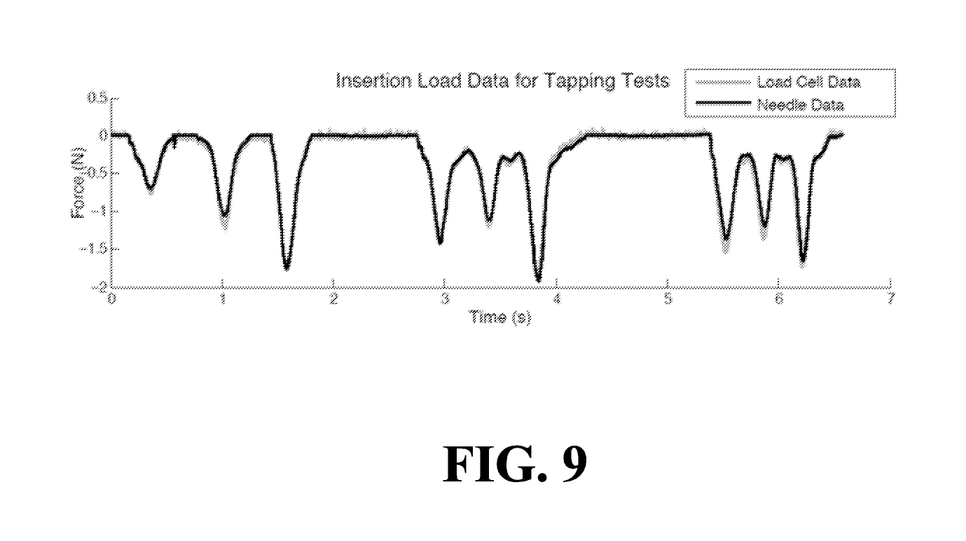

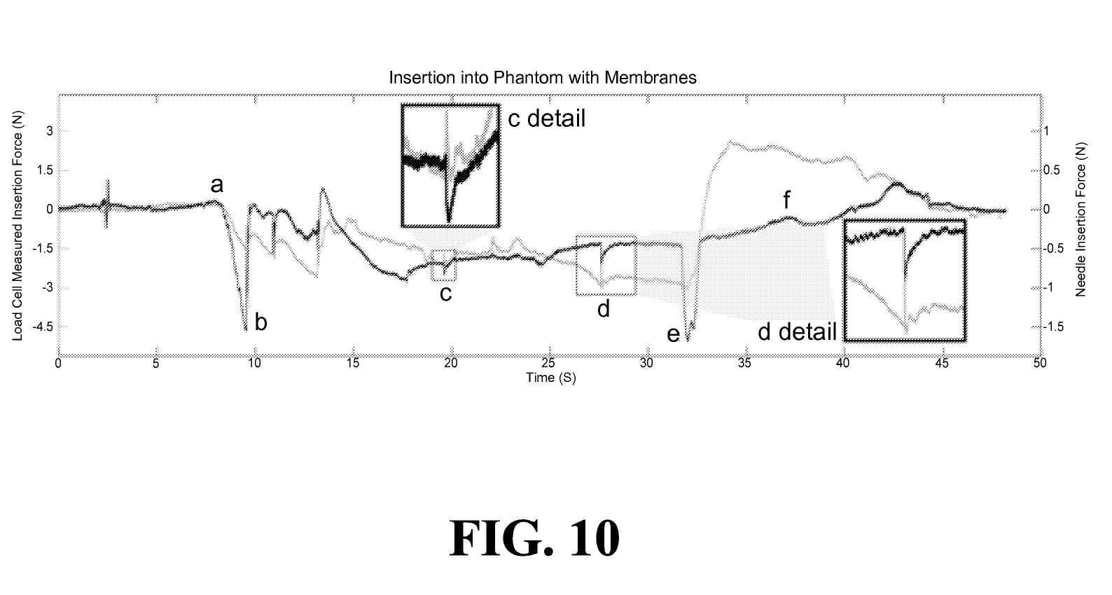

FIG. 9 shows example axial FBG data from a needle tip compared to force data from a needle base during tap testing, consistent with various aspects of the present disclosure. The recorded signals from the needle tip and the force/torque sensor at the needle base are nearly identical, with a lower noise floor in the case of the needle. This correspondence is to be expected as the tapping velocities were relatively low, so acceleration forces due to the mass of the needle did not significantly affect readings from the force/torque sensor in this case. A more interesting comparison is seen in FIG. 10.

FIG. 10 shows example axial FBG data from a needle tip compared to force data from a needle base during insertion into a tissue phantom; consistent with various aspects of the present disclosure. FIG. 10 shows (a) initial contact of needle and a phantom, (b) piercing through the first of three skin layers, (c) piercing first inner membrane, (d) piercing second inner membrane, (e) hitting a hard surface, and (f) extraction of the needle from a phantom. In this example, the needle was pushed through a PVC phantom (2:1 ratio of plastic and softener). The needle went through the phantom's skin, which included three layers of plastic and wax sheets, pierced two inner membranes, came in contact with a hard surface and then was completely extracted. As in the example shown in FIG. 9, the axial components of the needle and force/torque data are compared. Visible events in FIG. 10 are verified from video data and include membrane contact and puncture (b), hitting a hard surface (e), and exiting through membranes (f), which can be seen more clearly in the FBG data compared to the load cell. A tap was used to synchronize the F/T sensor, FBG and video data, and can be seen before (a) initial contact with the phantom.

The needle stylet tip is partially exposed outside the needle sheath, and one hole is partially visible outside the sheath. The tip forces experienced at the needle during insertion and piercing of the three-layer skin at (b) at times became larger than zero, and it is possible the needle undergoes some tensile effects as the sheath edge gets caught on a membrane. Similarly, during the retraction phase (f) of the needle, again the tip may be experiencing some tension while pulling on the inner membranes on its way out, hence a positive force reading is observed in the FBG data. Beyond the higher signal-to-noise ratio from the instrumented needle, a major difference is that the stylet is housed inside a sheath which slides against tissues producing friction forces that are transmitted to the needle base. The friction felt at the base masks the effects of small variations in the tip forces. Secondly, for sudden changes in velocity, the force sensor at the needle base experiences inertial forces due to the mass of the needle. The FBGs near the tip of the inner stylet do not experience either of these effects, and are therefore capable of discerning smaller dynamic forces at the tip.

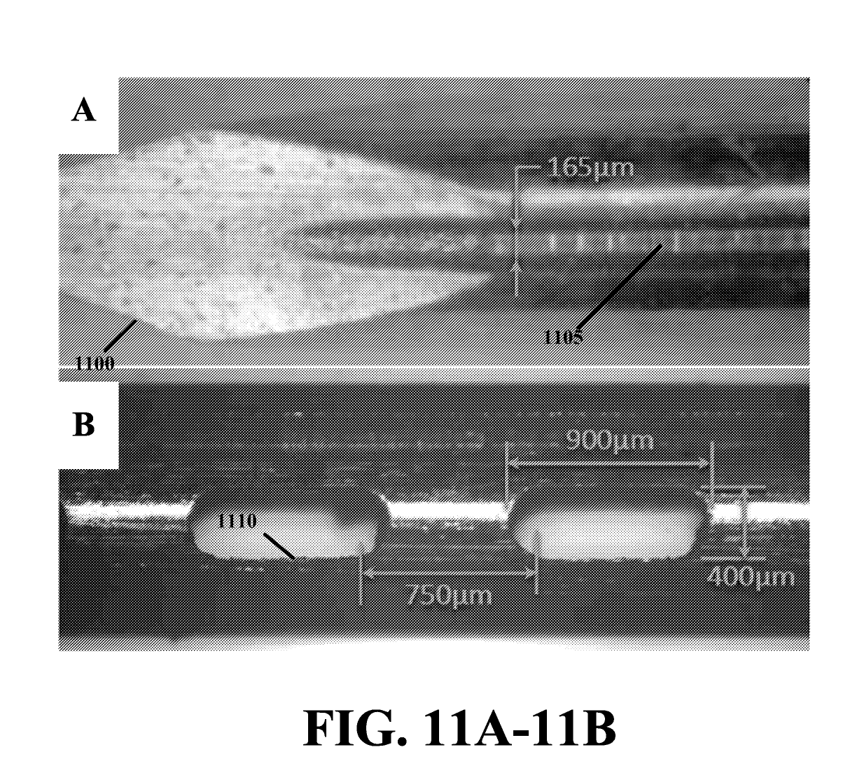

FIG. 11A shows an example microscope image of a needle tip 1100 and groove 1105 of a tissue-engagement apparatus, consistent with various aspects of the present disclosure. FIG. 11B shows an example microscope image of openings 1110 of a tissue-engagement apparatus, consistent with various aspects of the present disclosure. In certain embodiments, the tissue engagement apparatus needle includes two portions: a solid stylet and a removable exterior sheath. The stylet can be 1.008 mm in diameter, and the outer diameter and inner diameter of the outer sheath can be 1.270 mm and 1.066 mm respectively. The stylet holds the sensing elements, and is made of MP35N (a nickel-cobalt based alloy); the sheath is Inconel 625. MP35N in any heat-treated condition is particularly difficult to machine using traditional methods. Therefore, electric discharge machining (EDM) was used to create the grooves and holes, using a wire diameter of 80 μm. EDM also has no risk of shedding and embedding small ferromagnetic particles in the needle.

EDM can only be performed on metallic parts, thus the plastic standard luer-lock base was removed with a heat gun, and reattached after machining. After reassembly, the total metallic length of the needle from the plastic base was 147 mm. The total fiber diameter (core+cladding) is 125 μm, and FBG lengths are 5 mm. The fibers were adhered in the grooves using a medical grade epoxy. The sensor locations were at 31 mm, 81 mm, 131 mm and 141 mm from the plastic base. The sensors are set far enough apart to get a good approximation of the full curvature profile, and the middle of the last FBG set was centered over the holes to measure loads at the tip.

A method used in tip force calibration included applying known loads to the needle tip and monitoring the changes in the wavelength from each FBG, assuming that each FBG measures axial strains at its centroid, and that all FBGs experience the same strains as the needle material to which they are bonded. As noted, the FBGs are sensitive to temperature variations. To calibrate for temperature, the needle was placed in a controllable environmental chamber, with the temperature set between 15-45° C. Adequate time was allowed for the temperature to stabilize before each measurement. The linear relationship between wavelength and temperature was found for each sensor on the needle. Each gauge has a slightly different K_{T}, due to the FBG manufacture and its bond to the needle, and is dominated by the thermal expansion of MP35N (1.37e-5/° C.). The average value for K_{T} among the 12 FBGs was 0.023 nm/° C.

The expected wavelength shifts due to mechanical strains are comparable to those from temperature changes. Recall from equation 2 that for constant temperature Δλ_{B}=(1-Pₑ)ε_{z}λ_{B}. Given the strain found from FEA for an axial load of 1N (1.8e-5), and assuming a center wavelength of 1556 nm, a wavelength shift of 0.022 nm is expected at the upper FBG location. This means that the wavelength shift for a 1N axial load is similar to that for a temperature change of 1° C.

Assuming a uniform temperature for each triplet of FBGs along the needle length, variations in temperature should affect each FBG equally. However, as seen in FIG. 7, axial strain at the top FBG is greater than that of the lower FBGs due to the modified cross-section at the tip. Consequently, the effects of temperature and axial loading should be separable. However, to minimize effects of temperature variation on force calibration, loads were applied at a known frequency to the needle tip using a dual-mode lever arm system. The lever arm applies controlled forces with a resolution of 1 mN with a 0.2% force to signal linearity over a range of frequencies from 1 to over 200 Hz. The lever arm was connected to the needle tip with a short spring to apply tip loads in x, y or z, while the needle base was fixed. In the case of axial loading, the needle was held in tension to minimize strains due to bending. With the needle isolated in a foam-lined box, the dynamic force variations are easily distinguished from the much slower effects of ambient temperature variations.

In certain embodiments, the tissue-engagement apparatus includes seven holes, 0.5 mm long with 0.2 mm radius semi-circular edges, spaced 0.75 mm apart. The total length of the modified region is 8.4 mm. In cross section, the holes are positioned between the upper groove position and the other two grooves.

Additionally, for calibration, the lever arm was programmed to produce sinusoidally varying forces at 20 rad/s. The wavelength data from the needle were filtered using 10th order Butterworth filters to high pass frequencies above 2 Hz and low pass frequencies below 15 Hz. A peak detection algorithm was used to find the wavelength shifts for the corresponding applied loads. Loads varying from 0.005N to 0.05N in the x, y and z directions were tested.

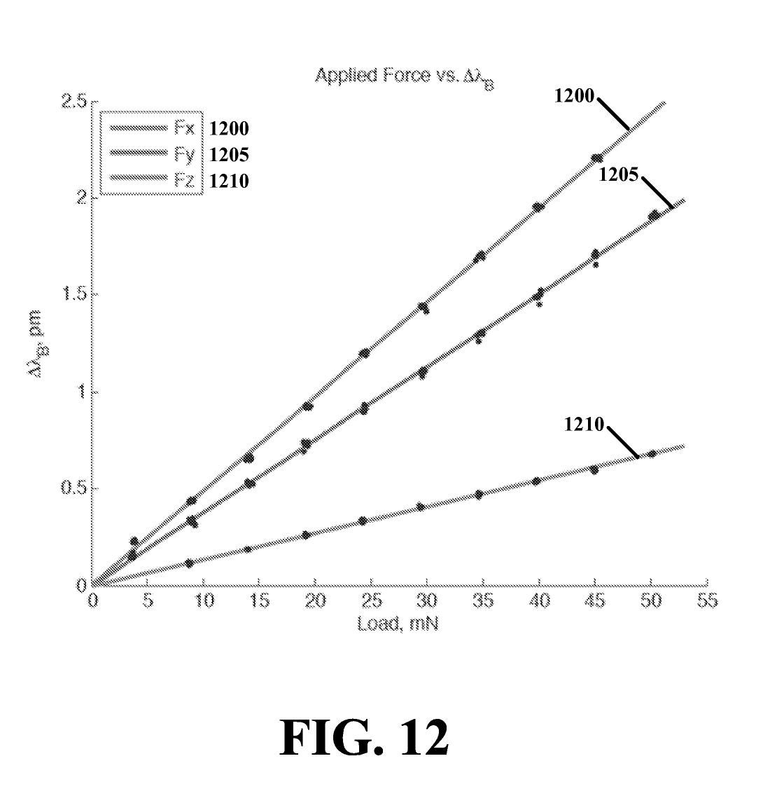

FIG. 12 shows an example plot of a wavelength shift to applied force for a FBG sensor at the needle tip of a tissue-engagement apparatus, consistent with various aspects of the present disclosure. The plot is a Δλ_{B} vs. Applied Force plot for one FBG sensor, number 12, at the tip of the needle. As shown in FIG. 12, this includes forces in the x-direction 1200, y-direction 1205, and z-direction 1210. Each point represents the difference between the minimum and maximum force over one period of the muscle arm during loading. FBG 12 is counter-clockwise from the top gauge (FBG 10) when viewed from the xy plane. Due to its placement, it is more sensitive to loads in the x-direction than in the y-direction.

Tests with the instrumented needle confirm basic predictions of the FEA. As seen in the calibration data in FIG. 12, the FBG wavelength shifts vary linearly with applied tip forces in the x, y and z directions. From tests with the lever arm, it was found that the minimum detectable forces with reasonable resolution, without filtering FBG data, are approximately 0.008N in the axial direction and 0.004N in the radial x and y directions. For the purposes of providing haptic feedback during minimally invasive surgery, the sensor response to small transient forces is of particular importance. Humans are sensitive to force variations in the range of tens to hundreds of Hz, with a peak sensitivity to vibrations around 250 Hz. For the case of needle manipulation in tissue, most frequencies of interest are in the tens of Hz, but when scraping hard or textured surfaces, vibrations with a frequency content of over 100 Hz are possible.

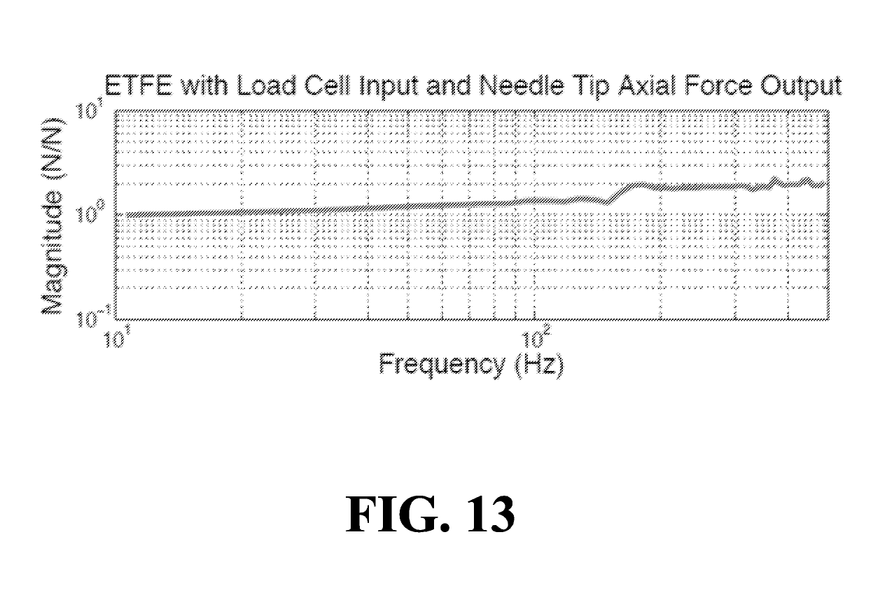

To test the frequency response of the needle and sensors, the needle was connected to a subwoofer, acting as a linear voice coil actuator, with a load cell at the center of its suspension pressing axially against the tip of the needle. The needle was adhered to the load cell through a small amount of polymer to prevent damage to the needle tip. A 5-500 Hz chirp signal was applied to the speaker through a function generator and amplifier, and data from the load cell and FBG sensors were collected. The transfer function between the load cell and the average response over the tip 3 FBGs was obtained using the ETFE (empirical transfer function estimation) method. The frequency range was split into 45 equally spaced bins and the transfer function was averaged across the bins and multiple samples to minimize noise. As seen in FIG. 13 the frequency response of the needle is nearly flat over the range tested, with some increase in amplitude above 200 Hz, likely due to a small amount of bending that occurred at these higher frequencies.

For further details regarding tissue-engagement apparatuses, reference is made to U.S. Provisional Patent Application Ser. No. 61/772,061, to which this document claims priority benefit of, filed on Mar. 4, 2013; this patent document and its accompanying Appendices are fully incorporated herein by reference.

Various embodiments described above, and shown in the figures may be implemented together and/or in other manners. One or more of the items depicted in the present disclosure can also be implemented in a more separated or integrated manner, or removed and/or rendered as inoperable in certain cases, as is useful in accordance with particular applications. In view of the description herein, those skilled in the art will recognize that many changes may be made thereto without departing from the spirit and scope of the present disclosure.

1. A tissue-engagement apparatus comprising:
a distal needle portion having a sharp-end region configured and arranged to be applied to a tissue surface;
a proximate needle portion configured and arranged to attach to a needle base; and
an elongated needle portion, situated between the distal needle portion and the proximate needle portion, having
a plurality of openings configured and arranged to accentuate haptic-type forces carried by the elongated needle portion in response to engagement between the sharp-end region and the tissue surface, and
a communication pathway configured and arranged to convey information, from the distal needle portion along the elongated needle portion, that characterizes forces due to the engagement between the sharp-end region and the tissue surface.

2. The tissue-engagement apparatus of claim 1, wherein the distal needle portion and the elongated needle portion are configured and arranged as contiguous parts of a needle, and wherein the communication pathway is configured and arranged to secure at least one fiber optic line, the fiber optic line being configured and arranged to convey optical information, and further comprising the needle base, at least one Fiber Bragg grating (FBG) sensor configured and arranged along the elongated needle portion, the at least one FBG sensor being configured and arranged with the at least one fiber optic line to convey at least one of transverse load information, axial load information, and temperature information.

3. The tissue-engagement apparatus of claim 1, further including at least one sensor configured and arranged to measure optical wavelength shifts, communicated by the communication pathway, that occur due to the forces at the sharp-end region due to a compressive load on the sharp-end region and the elongated needle portion due to engagement between the sharp-end region and the tissue surface.

4. The tissue-engagement apparatus of claim 1, wherein the information conveyed by the communication pathway includes temperature inside the tissue surface, and three-dimensional quantification of bending, and tensile and compressive forces along the length of the elongated needle portion.

5. The tissue-engagement apparatus of claim 1, wherein the sharp-end region is a trocar tip, and the tissue-engagement apparatus is compatible with magnetic resonance imaging (MRI) and the communication pathway defines one or more grooves constructed along the elongated needle portion, with each of said one or more grooves securing a fiber optic line.

6. The tissue-engagement apparatus of claim 1, further including at least one groove in and along the elongated needle portion between the distal end portion and the proximate needle portion, at least one groove being configured and arranged to house the communication pathway.

7. The tissue-engagement apparatus of claim 1, wherein the communication pathway is configured and arranged to convey information indicative of a vibration of the sharp-end region.

8. The tissue-engagement apparatus of claim 1, wherein the plurality of openings are located closer to the distal needle portion than to the proximate needle portion, and the sharp-end region is configured and arranged to puncture the tissue surface, and the communication pathway is configured and arranged to convey information including information indicative of a vibration of the elongated needle portion and light.

9. A method comprising:
providing a distal needle portion having a sharp-end region configured and arranged to be applied to a tissue surface, a proximate needle portion configured and arranged to attach to a needle base; and an elongated needle portion, situated between the distal needle portion and the proximate needle portion, having a plurality of openings configured and arranged to accentuate loads carried by the elongated needle portion in response to engagement between the sharp-end region and the tissue surface, and a communication pathway configured and arranged to convey information, from the distal needle portion along the elongated needle portion, that characterizes forces due to the engagement between the sharp-end region and the tissue surface; and
applying the sharp-end region to the tissue surface.

10. The method of claim 9, wherein applying the sharp-end region to the tissue surface includes determining the information via at least one Fiber Bragg grating (FBG) sensor configured and arranged along the elongated needle portion and communicatively coupled to the communication pathway.

11. The method of claim 10, wherein determining the information includes sensing loads at the sharp-end region using the at least one FBG sensor.

12. The method of claim 10, wherein determining the information includes sensing temperature at the sharp-end region using the at least one FBG sensor.

13. The method of claim 9, further including conveying the information, wherein the information includes the forces on the sharp-end region during application of the sharp-end region to the tissue surface along a longitudinal portion and cutting loads at the sharp-end region.

14. The method of claim 9, wherein applying the sharp-end region to the tissue surface includes determining the information via at least one sensor centered over at least one of the plurality of openings.

15. The method of claim 10, wherein the communication pathway includes an optical fiber, the method further including:
providing a light along the optical fiber and, thereby, transmitting light through the at least one FBG sensor;
sensing optical wavelength shifts in response to the providing light using the at least one FBG sensor.

16. The tissue-engagement apparatus of claim 1, wherein the communication pathway includes a communication line and is configured and arranged to secure at least one fiber optic line, the fiber optic line being configured and arranged to convey optical information, further including at least one Fiber Bragg grating (FBG) sensor, the at least one FBG sensor being configured and arranged with the at least one fiber optic line to convey at least one of transverse load information, axial load information, and temperature information.

17. The tissue-engagement apparatus of claim 16, wherein the FBG sensor is configured to convey the information by sensing optical wavelength shifts, communicated by the communication pathway, that are due to the forces at the sharp-end region due to a compression load on the sharp-end region, and the elongation needle portion due to engagement between the sharp-end region and the tissue surface.

18. The tissue-engagement apparatus of claim 16, wherein the at least one FBG sensors includes a plurality of FBG sensors located at various locations of the tissue-engagement apparatus, the plurality of FBG sensors configured and arranged to convey the optical information to approximate a curvature profile of the tissue-engagement apparatus.

19. The tissue-engagement apparatus of claim 18, wherein at least one of the plurality of FBG sensors is centered over the openings, and configured and arranged to sense forces at the sharp-end region.

20. The tissue-engagement apparatus of claim 1, wherein the communication pathway is configured and arranged to secure a plurality of fiber optic lines, the plurality of fiber optic lines being configured and arranged to convey optical information and the plurality of fiber optic lines being embedded a threshold degree apart.
